(19) United States
(12) Patent Application Publication
(10) Pub. No.: US 2011/0152317 A1
(43) Pub. Date: Jun. 23, 2011

(54) PREVENNTING THE OCCURRENCE OF OBESITY

(75) Inventor: Zhen Yan, Charlottesville, VA (US)

(73) Assignee: DUKE UNIVERSITY, Durham, NC (US)

(21) Appl. No.: 12/996,654

(22) PCT Filed: Jul. 9, 2009
(86) PCT No.: PCT/US2009/003996

§ 371 Date: Feb. 23, 2011

Related U.S. Application Data
(60) Provisional application No. 61/129,714, filed on Jul. 14, 2008.

(51) Int. Cl. A61K 31/4706; A61P 3/04

(52) U.S. Cl. 514/313; 514/311

(57) ABSTRACT

Administration of autophagy inhibitor (e.g. 400 mg chloroquine daily) prevents occurrence of obesity and insulin resistance in human subject on prolonged high-fat diet.

### CROSS-REFERENCES TO RELATED APPLICATIONS

This application claims the benefit of U.S. Provisional Application No. 61/129,214, filed Jul. 14, 2008, the whole of which is incorporated herein by reference.

### TECHNICAL FIELD

This invention is directed to drug administration to prevent the occurrence of obesity in mammal (e.g. human) subjects, despite a high-fat diet.

### BACKGROUND OF THE INVENTION

Obesity is severe excess body fat. Diagnosis is based on body mass index (BMI) which is calculated by dividing mass in kilograms by height in meters squared, and also on waist circumference. As used herein obesity means a BMI>30.0 kg/m² and/or a waist circumference of greater than 40 inches for men and greater than 35 inches for women.

During the past twenty years there has been a dramatic increase in obesity in the United States. In 2006, only four states had a prevalence of obesity less than 20%. There are a number of diseases that are induced/exacerbated by obesity. These include Type 2 diabetes, cardiovascular disease, stroke, hypertension, hypothyroidism, dyslipidemia, hyperinsulinemia, insulin resistance, glucose intolerance, congestive heart failure, angina pectoris, cholecystitis, cholelithiasis, osteoarthritis, gout, fatty liver disease, sleep apnea and other respiratory problems, polycystic ovary syndrome (PCOS), fertility complications, pregnancy complications, psychological disorders, uric acid nephrolithiasis (kidney stones), stress urinary incontinence, and cancer of the kidney, endometrium, breast, colon, rectum, esophagus, prostate and gall bladder.

Because of the disease risks associated with it, it is important that development of obesity be avoided. To that end, a myriad of diets have been proposed. For example, high-fat diets (more than 40% of calories from fat) are to be avoided. This means restrictions on eating fried foods, red meat, and fat based products (e.g., ice cream), which most people find good-tasting.

It is an object herein to prevent the occurrence of obesity despite a high-fat diet (e.g. more than 40% of calories from fat, e.g. 40 to 70% of calories from fat, e.g. 60% of calories from fat.).

### SUMMARY OF THE INVENTION

The invention in one embodiment herein is directed at a method for preventing (countering in advance) the occurrence of obesity in a subject ingesting a high-fat diet (method whereby obesity does not occur in a subject ingesting a high fat diet), comprising administering to said subject an adipogenesis (formation of fat or fatty tissue) impeding amount of an autophagy inhibitor.

The invention in another embodiment herein is directed to preventing regain of body weight in a subject following diet, exercise and/or other interventions (e.g. bariatric surgery) that lead to loss of body weight and reduction of obesity comprising administering to said subject an adipogenesis impeding amount of an autophagy inhibitor.

### DETAILED DESCRIPTION

A test for determining what compounds are autophagy inhibitors that are effective in inhibiting adipogenesis is as follows. Mouse 3T3 pre-adipocytes (available from the American Type Culture Collection) grown to 80% confluence are incubated with BSA-conjugated palmitate (e.g. available from Sigma) in Dulbecco's Modified Eagle's Medium, with the BSA-conjugated palmitate being present in a concentration of 0.4 mM, together with putative autophagy inhibitor with increasing concentration starting at 1 mM for various durations starting at 24 hours. If a putative inhibitor is potent in inhibiting adipogenesis, lipid droplet accumulation is significantly reduced.

Test results in the above-described test show that compounds are autophagy inhibitors, i.e. that are effective in inhibiting adipogenesis in mice and an assumption is made that these same compounds will be effective in inhibiting adipogenesis in humans.

Once an effective compound is determined, mice on high fat diets are administered by oral gavage or other routes, such as intraperitoneal injection, that compound in selected amounts and amount effective to prevent occurrence of obesity (i.e the amount where obesity does not occur) is determined. What constitutes the presence of obesity and insulin resistance for mice is indicated in Background Example 1 below.

The next step is to convert amount found effective so that obesity does not occur in mice on high fat diets to that effective for humans on high fat diets. This is done by multiplying the amount determined to be effective for mice by 70 kg (average body weight of humans) divided by the weight of a mouse administered as in the paragraph directly above. Consultation with a toxicologist and experiments in mice for toxicological analysis is then done to determine dosages for humans.

Autophagy inhibitors for use herein and useful dosages therefor where indicated, are chloroquine (5-10 mg/kg per day, roughly 350-750 mg daily), hydroxychloroquine (Plaquenil™) 400-600 mg daily), primaquine (15 mg daily), bafilomycin Al (e.g. 10 mM in blood), okadaic acid (1-100 mM in blood), 4-amino-4-imidazole carboxamide riboside (AICAR) (100-200 mg/kg), adenosine (6-12 mg IV), vinblastine (0.3 mg/kg IV), wortmannin (4 mM in blood), N6-mercaptopurine riboside (0.3 mM in blood), 3-methyladenine, autophagy-suppressive algal toxins which inhibit protein phosphatases of type 2A or type 1, analogues of cAMP, and drugs which elevate cAMP levels. In addition, antisense or siRNA that inhibits expression proteins essential for autophagy e.g. siRNA of genes essential or for autophagy, e.g. siRNA or Atg 5, Atg 6, Atg 7, Cathespin D, Lamp 1 and Lamp 2, are useful.

Routes of administration in general are oral administration, rectal administration, intraperitoneal injection, subcutaneous injection, intramuscular injection and intravenous injection. In some cases, the route of administration is indicated in the paragraph directly above. Preferred route of administration for chloroquine, hydroxychloroquine and primaquine is oral.

The following Background Examples and Working Example illustrate the functionality of autophagy inhibitors and administration to a human on a high-fat diet without obesity occurring.

### Background Example 1

### Demonstration in a Mouse Model that a High-Fat Diet Induces Obesity and Insulin Resistance

Five adult (12 week old) male C57BL/6 mice (The Jackson Laboratory, Bar Harbor, ME) were subjected to 12 weeks of high-fat diet (HF) (60% of calories from fat) with a separate group of five mice on normal chow (NC) for the same duration as control. A significant difference in body weight between NC and HF mice was detected 4 weeks after implementation of the high-fat diet, and the body weight of the HF group continued to increase to about 55.4% above the value in age matched NC mice after 12 weeks of the high-fat diet. Dual-energy X-ray absorbtiometry analysis (DEXA) showed significantly increased percent body fat in HF v. NC mice. The development of severe obesity in HF mice was accompanied by 89.0% increase (P<0.001) in blood glucose area under curve (AUC) during a glucose tolerance test. These findings indicate that high-fat diet in adult mice induced severe obesity and insulin resistance.

### BACKGROUND EXAMPLE 2

### Chloroquine Injection Blocked Occurrence of Obesity Caused by High-Fat Diet

Five of the same kind of non-obese mice as used in Background Example 1 on a high-fat diet (60% of calories from fat) were injected with chloroquine (60 mg/kg, intraperitoneal) daily for 12 weeks of high-fat diet feeding. The injections blocked the occurrence of obesity and insulin resistance (as determined in a glucose tolerance test) and blocked high-fat diet induced changes in skeletal muscle as determined by mass spectrometry-based profiling of lipid species.

### Working Example I

### Autophagy Inhibitor Injection Prevents Occurrence of Obesity and Insulin Resistance in Human on High-Fat Diet

A sixty year old man with BMI of 20 eats a diet where 50% of calories come from fat, e.g. cheeseburgers or steak, onion rings or french fries, and ice cream or pecan pie for dessert at dinner, cold cut sandwich and fries or pizza for lunch, eggs, sausage, hash browns for breakfast. The subject also orally takes 400 mg chloroquine orally daily. The regimen is continued for 12 weeks. The subject continues to have BMI of 20 and does not develop insulin resistance.

### Variations

The foregoing description of the invention has been presented describing certain operable and preferred embodiments. It is not intended that the invention should be so limited since variations and modifications thereof will be obvious to the skilled in the art, all of which are within the spirit and scope of the invention.

What is claimed is:

1. A method for preventing the occurrence of obesity in a subject ingesting a high-fat diet, comprising administering to said subject an adipogenesis impeding amount of an autophagy inhibitor.

2. The method of claim 1 where the autophagy inhibitor is selected from the group consisting of chloroquine, hydroxychloroquine and primaquine and the route of administration is oral.

3. A method for preventing regain of body weight in a subject following diet, exercise and/or other interventions that lead to loss of body weight and reduction of obesity comprising administering to said subject an adipogenesis impeding amount of an autophagy inhibitor.

4. The method of claim 3 where the autophagy inhibitor is selected from the group considering of chloroquine, hydroxychloroquine and primaquine and the route of administration is oral.
